(19) United States
(12) Patent Application Publication
(10) Pub. No.: US 2015/0299648 A1
(43) Pub. Date: Oct. 22, 2015

(54) IMPROVED CULTIVATION MEDIA AND PROCESS FOR IMPROVED PROTEIN PRODUCTION BY PICHIA STRAINS

(71) Applicant: MERCK SHARP & DOHME CORP., Rahway, NJ (US)

(72) Inventors: Sehoon Kim, Hanover, NH (US); Marc D'Anjou, Enfield, NH (US); Muralidhar R. Mallem, Lebanon, NH (US); Ishaan Shandil, Nashua, NH (US); Adam Nylen, Enfield, NH (US); Nathan Sharkey, Plainfield, NH (US); Seemab S. Shaikh, Lebanon, NH (US)

(73) Assignee: Merck Sharp & Dohme Corp, Rahway, NJ (US)

(21) Appl. No.: 14/647,874

(22) PCT Filed: Nov. 22, 2013
(86) PCT No.: PCT/US13/71372

§ 371 Date: May 28, 2015

Related U.S. Application Data
(60) Provisional application No. 61/731,166, filed on Nov. 29, 2012.

(51) Int. Cl. C12N 1/16; C07K 16/00; C07K 14/62

(52) U.S. Cl. CPC C12N 1/16; C07K 14/62; C07K 16/00

(57) ABSTRACT

The present invention provides optimized cell culture media and fed-batch cultivation processes to improve the viability and volumetric production of heterologous proteins in Pichia. The disclosed media and processes utilize a non-fermentable sugar or sugar alcohol as an osmoprotectant to improve the robustness of Pichia production strains during methanol inducible fermentation.

### FIELD OF THE INVENTION

The present invention relates to process technology developed to improve the robustness of Pichia strains used for the production of heterologous proteins of interest in methanol inducible fermentation systems.

### BACKGROUND OF THE INVENTION

The methylotrophic yeast Pichia pastoris is a commonly used microbial host cell in the biopharmaceutical industry for the production of a variety of heterologous recombinant proteins (Ellis, 1985; Vozza 1996; Darly and Hearn, 2005; Catena 2011; Stergiou 2011) however high levels of production typically require some degree of process optimization. The Pichia expression system is particularly useful in cases when Escherichia coli protein synthesis fails to deliver correctly folded proteins and Saccharomyces cerevisiae glycosylation patterns result in inactive hyperglycosylated proteins. The FDA approval of therapeutic proteins produced in Pichia and the availability of glycoengineered strains capable of producing heterologous proteins predominantly as single glycoforms may pave the way for the mainstream use of Pichia production platforms for the commercial production of biopharmaceutical glycoproteins.

Despite the current availability of five distinct protein production platforms (bacteria, yeasts, plants, insect cells and mammalian cells) Pichia expression systems are becoming increasingly popular alternatives to mammalian expression systems due to several advantageous features including: desirable host-vector system features including the tightly regulated and highly inducible alcohol oxidase 1 (AOX1) promoter, site-specific target gene integration, reasonable transformation efficiencies, genetic stability of the exogenous sequences during continuous and large-scale fermentation, and the abilities to direct the extracellular secretion of the product and to produce properly folded proteins with correct disulfide bond formation. As a simple eukaryote, Pichia, is capable of many of the same posttranslational modifications, including, methylation, acylation, proteolytic processing, O- and N-linked glycosylation, and targeting to subcellular compartments typically associated with the use of higher eukaryotic host cells. In addition, the fact that the P. pastoris genome has recently been sequenced provides an opportunity to employ systems biology strategies for production strain improvements. Therefore, the decision to use Pichia as a protein production platform could translate into reduced drug development timelines, due to the ability to exploit the rapid cell cycle time, ease of genetic manipulation, and the potential for strain improvement aspects of a Pichia expression system.

As a protein expression system, P. pastoris provides the advantages of a microbial system characterized by the potential for achieving high cell densities during fermentation. Capacity for high cell density growth is especially important for the production of secreted proteins, because the concentration of product in the medium is expected to be roughly proportional to the concentration of cells in culture. This is attributed to the fact that P. pastoris secretes only low levels of endogenous host cell proteins and virtually no proteases. Therefore, the vast majority of the total protein present in the harvested growth media is the secreted heterologous protein that the strain has been engineered to produce. Generally speaking, expression levels reported in the literature for the production of recombinant proteins Pichia systems are highly variable and range from the milligrams-per-liter to gram-per-liter levels (d'Anjou and Daugulis 2000).

Pichia cultures produce high cell densities on inexpensive, defined media using well-developed fermentation protocols, considerations which provide an opportunity to lower the production cost of recombinant glycoproteins. Suitable culture media provides pure carbon sources (glycerol and methanol), biotin, salts trace elements and water. The requisite media components are noticeably free of undefined ingredients which can be the source of pyrogens or toxins making Pichia expression systems particularly suitable for the production of human pharmaceuticals for both clinical studies and commercial purposes.

Glycoengineered Pichia pastoris strains can successfully express and secrete recombinant therapeutic proteins with humanized N-glycosylation including monoclonal antibodies (Hamilton and Gerngross, 2007; Potgieter, 2009). However, extensive genetic modifications of Pichia strains can cause fundamental changes in cell wall structures which can compromise the robustness of some glycoengineered strains by predisposing the cell to lysis during fermentation. The integrity of yeast cell structure is the main determinant of cell robustness, which is in turn a critical requirement to achieve high cell density cultivation of Pichia production strains for extended times of methanol induction. In practice, certain glycoengineered strains have been observed to have a marked increase in intracellular protease leakage into the fermentation broth leading to reduced cell viability and undesirable effects on product yield and quality. Therefore, improved culture conditions utilizing optimized media and fermentation conditions designed to improve the robustness of engineered Pichia strains are of value and interest to the field.

### SUMMARY OF THE INVENTION

In one embodiment the invention provides a cell culture medium optimized for use in a methanol inducible fermentation system (under the control of the AOX1 promoter) for the production of a protein of interest in yeast host cells using a fed-batch fermentation process wherein the fermentation medium comprises a basal medium supplemented with a non-fermentable sugar or a non-fermentable sugar alcohol as an osmoprotectant. The media can be used to improve the robustness of glycoengineered strains of yeast during the methanol induction phase of suitable fermentation protocols. Alternatively, the media can also be used to improve the integrity and viability of wild-type Pichia production strains under substrate-limited fed-batch conditions characterized by long induction times (ie., longer than 10 days).

In particular embodiments, the osmoprotectant can be selected from maltose, sorbose, ribose, maltitol, myo-inositol, mellibiose, and quinic acid and is present at concentration is about 25 g/L to about 50 g/L. The presence of the osmoprotectant in the batch media should increase and maintain the osmolality of the batch media more than about 50 mOsm/kg for compared to the osmolality of a fed-batch culture of the same host cell in culture media not supplemented with an osmoprotectant. In practice, the reasonable range of induction media osmolality which will be suitable for maintaining cell fitness (e.g., robustness) of a Pichia production strain is 450-900 mOsm/kg. In order to significantly improve the robustness of a Pichia producton strain the increased osmolality of the induction media should be maintained at a higher level of 450 mOsm/kg for at least 24 hours period of time. In some embodiments the osmolality of the supplement induction media will be at least about 460, 470, 475, 480, 490, 500, 510, 520, 530, 540, 550, 560, 570, 580, 590 or 600 mOsm/kg. The length of time which the increased osmolality is maintained will vary from at least about 24 hours until completion of the methanol induction phase (e.g., ranging from about 24 to about 100 hours).

The disclosed osmoprotectants can be added to any suitable basal medium. For example, as shown herein, when the production strain is a glycoengineered strain of Pichia pastoris, a suitable basal medium is BSGY and a suitable osmoprotectant is the sugar alcohol maltitol (4-O-α-glucopyranosyl-D-sorbitol) or the non-fermentable sugar maltose (also referred to as maltobiose). In particular embodiments the osmoprotectant can be added in addition to other media supplements, including, but no limited to mixes comprising amino acids, vitamins, trace metals or basal salts.

Another embodiment of the invention provides a methanol fed-batch fermentation medium comprising an osmoprotectant selected from a nonfermentable sugar and a nonfermentable sugar alcohol. In particular embodiments, the invention provides methanol fed-batch fermentation medium comprising an osmoprotectant selected from maltose, sorbose, ribose, maltitol, myo-inositol, mellibiose, and quinic acid and is present at a concentration of about 25 g/L to about 50 g/L. To obtain a maximum osmoprotective effect, the non-fermentable sugar or non-fermentable sugar alcohol should be included in the culture medium at an optimal concentration.

In alternative embodiment, the invention provides a method of improving the volumetric productivity of a glycoprotein of interest in a yeast fermentation culture comprising: a) providing a glycerol fed-batch yeast host cell culture comprising high density yeast cells that contain a gene encoding a polypeptide of interest, which gene is expressed under conditions of fermentation, b) providing a methanol fed-batch medium containing an osmoprotectant, and c) inducing the yeast host cells under fermentation conditions that allow expression of the recombinant protein wherein the volumetric productivity of the protein of interest is higher than the productivity obtained using identical fermentation conditions to produce the same glycoprotein in medium that lacks the osmoprotectant. The improved productivity is achieved without having an adverse effect on the cell survival and growth. Inclusion of the osmoprotectant during the methanol induction phase, has no effect on the presence of undesirable host cell proteins or on the product quality including but not limited to the N-glycosylation of target protein.

In one embodiment, the maximum titer of heterologous protein (e.g., anti-Her2 monoclonal antiby) obtained in the presence of culture media comprising the non-fermentable sugar or sugar alcohol (e.g., 5% maltitol) at 600 mOsm/kg in induction phase was increased by 120-% over that obtained in a control culture with physiologic osmolality. Another target therapeutic protein (e.g., insulin precursor) was not detectable due to cell lysis without supplementation of osmoprotectants but intact protein was produced in the presence of osmoprotectant (e.g., 2% maltitol) at 83 h of post-induction.

In particular embodiments, the Pichia host cells are production strains that have been glycoengineered to produce a therapeutic protein and the strain: a) includes a nucleic acid that encodes an alpha-1,2-mannosidase that has a signal peptide that directs it for secretion, b) comprises a nucleic acid sequence that encode one or more glycosylation enzymes or oligosaccharyltransferases; c) comprise a disruption or deletion of one or more of a functional gene product encoding an alpha-1,6-mannosyltransferase activity, mannosylphosphate transferase activity, a β-mannosyltransferase activity or a dolichol-P-Man dependent alpha(1-3) mannosyltransferase activity, or d) produces glycoproteins that have predominantly an N-glycan selected from the group consisting of complex N-glycans, hybrid N-glycans, and high mannose N-glycans. Examplary therapeutic protein can be selected selected from the group consisting of kringle domains of the human plasminogen, erythropoietin, cytokines, coagulation factors, soluble IgE receptor α-chain, IgG, IgG fragments, IgM, urokinase, chymase, urea trypsin inhibitor, IGF-binding protein, epidermal growth factor, growth hormone-releasing factor, annexin V fusion protein, angiostatin, vascular endothelial growth factor-2, myeloid progenitor inhibitory factor-1, osteoprotegerin, α-1 antitrypsin, DNase II, α-feto proteins, insulin, Fc-fusions, and HSA-fusions.

In an alternative embodiment the invention provides a method for producing glycoprotein compositions in wild type Pichia sp or glycoengineered host cells using a methanol inducible fermentation system which utilizes a methanol fed-batch fermentation medium comprising an osmoprotectant selected from a nonfermentable sugar and a nonfermentable sugar alcohol. Nonfermentable sugars and sugar alcohols such as maltose, sorbose, ribose, maltitol, myo-inositol, mellibiose, and quinic acids are newly identified as osmoprotectants useful for increasing and maintaining osmlality to protect Pichia cells, and in particular engineered Pichia cells from lysis under hypo-osmotic condition, which results from depletion or limitation of substrates in the medium during methanol induction phase in high-cell-density cultures.

In another alternative embodiment, the invention provides a method of improving the cell viability of engineered Pichia strains comprising: a) providing a high density Pichia cell culture wherein the cells contain a gene encoding a polypeptide of interest, which gene is expressed under conditions of fermentation, b) providing a methanol fed-batch medium containing an osmoprotectant, and c) inducing the Pichia cells under fermentation conditions that allow expression of the recombinant protein wherein the cell viability of the Pichia cells is greater than the viability of identical Pichia cells cultured under identical fermentation conditions in medium lacking the osmoprotectant. In particular embodiments of the invention the osmoprotectant can be selected from a nonfermentable sugar and a nonfermentable sugar alcohol. More specifically, the osmoprotectant can be selected from maltose, sorbose, ribose, maltitol, myo-inositol, melibiose, and quinic acid present at concentration is about 25 g/L to about 50 g/L.

### BRIEF DESCRIPTION OF THE DRAWINGS

FIGS. 1A-1B: FIG. 1A shows a graph illustrating the correlation of osmolality with respect to concentration of sorbitol. FIG. 1B is a bar graph showing the osmolalities of four tested sugars or sugar alcohols (maltitol, sorbitol, glycerol and glucose) dissolved in water at 500 mM concentration.

FIGS. 2A-2F: FIGS. 2A-2F summarize the results of screening assays performed to evaluate sugars and sugar alcohols as osmoprotectants using P. pastoris (yGLY21058) producing glycosylated insulin precursor (GIP). FIGS. 2A and 2E provides SDS-PAGE analysis for 10 μL of supernatant taken from cultures comprising various sugar or sugar alcohols. M denotes pre-stained molecular weight markers and the arrow(s) indicate the position of the desired heterologous protein product. FIGS. 2B and 2E provide a graphic representation of data which provides an index of cell lysis (mg DNA/L) after 80 hours of induction. FIGS. 2C and 2E provide a graphic representation of the osmolality (mOsm/kg H2O) of the induction media after 80 hrs.

FIG. 3: shows a graph of consumption profiles of sorbitol and maltitol during the fed-batch fermentation of P. pastrois. Profiles for independent runs are shown.

FIGS. 4A-B show graphs illustrating the effect of maltitol with or without sorbitol on cell robustness: FIG. 4A illustrates cell growth (%)=[Cell(t)]/[Cell(0)]×100. FIG. 4B shows the index of cell lysis (mg DNA/L).

FIGS. 5A-D: show graphs of fermentation profiles of P. pastoris strain in BSGY (Control media without further supplements) and test strains cultured with supplements comprising Mix 2 (amino acids, trace metals, basal salts, and vitamins) or Maltitol, or Maltitol+Mix2 in BSGY media. FIG. 5A is a graph showing Cell density (gWCW/L), FIG. 5B is a graph showing relative production of an exemplary antibody [mg Ab (t)/mg Ab (t=120 h of induction) of yGLY13979 in control], (FIG. 5C is a graph showing the index of cell lysis (mg DNA/L), and FIG. 5D is a graph showing osmolality (mOsm/kg H₂O) for each strain.

### DETAILED DESCRIPTION OF THE INVENTION

Methanol inducible fermentation systems based on the AOX1 promoter are based on the use of glycerol as a substrate for biomass growth, followed by a methanol feed for induction. Generally, a multistage fermentation process including a glycerol batch phase, glycerol fed-batch phase, transition phase, and methanol induction phase is employed for the production of recombinant proteins using a Pichia production strain. During the first stages, the cells are cultured in a glycerol-containing medium, which is the carbon source that is most commonly used to accumulate biomass. The second production phase typically introduces a fed-batch transition phase in which glycerol is fed to the culture at a rate-limiting rate (0.005-0.1/h of specific growth rate) for 8-12 h in order to further increase the biomass and to prepare the cells for the upcoming induction phase. The final stage, which is the induction phase, is initiated by the gradual addition of methanol to the culture. The methanol can be continuously fed either at constant rate or at exponential rate under carbon-limited condition or bolus added at DO-spike indicating substrate depletion under oxygen-limited condition. The durations of each growth phase are typically 30±5 h at the batch growth phase depending on the strain and initial concentration of carbon source, 8-12 h at glycerol fed-batch phase, and 48-200 h at methanol induction phase depending on the fitness of Pichia strains.

Methanol is utilized not only as inducer for target protein production but also as carbon source for cell growth. The specific productivity of target protein is influenced by many factors including genetic engineering of strain (e.g., gene constructs and plasmid copy number) and culture process (e.g., culture media and fermentation methods). The volumetric productivity of target protein depends on the cell density and secretion efficiency. In order to maximize the volumetric productivity (titer), P. pastoris is cultivated in high-cell density using fed-batch methods. Typically the fed-batch culture consists of three growth phases: glycerol batch culture, first fed-batch using glycerol, and second fed-batch using methanol. Each growth phase shows different growth kinetics, which is influenced by media components and feeding protocols (e.g., carbon-limited and oxygen-limited methods). The addition of methanol and/or other carbon sources need to be well controlled to minimize the formation of by-product (e.g., lactate; acetic acid), cell lysis, and repressive effects of known (e.g., glucose; glycerol) and unknown metabolites.

In any given expression vector system, the titer (i.e. amount of desired protein product produced) is largely dependent upon cell growth and feeding methods of primary carbon sources including the use of methanol as an inducer. Generally speaking, the robustness of a glycoengineered P. pastoris production strain decreases as fermentation time increases, under both carbon limited and dissolved-oxygen-limited high-cell density fed-batch cultures. While not wishing to be bound by a particular theory, decreased Pichia robustness is most likely attributed to increased rates of host cell lysis in response to the hypo-osmotic and hypoxic stresses which occur during the induction phase of Pichia cultivation.

Early investigators reporting studies designed to enhance the production of a given recombinant protein in Pichia concluded that expression levels were largely dependent on either inherent features of the protein such as its amino acid sequence, its tertiary structure or on the site of integration of the expression vector. It is now appreciated that the level of expression of a heterologous protein in Pichia depends on other factors as well including, the nature of the signal sequence used, the number of copies of the gene which are integrated into the chromosome, composition of the culture media and the fermentation conditions that are used for growth and induction.

Typically process optimization efforts reported in the literature include optimization of expression conditions such as temperature, dissolved oxygen, cell density, media composition, pH and methanol feed rate. Potgieter et al. (2009) reported that process variables such as pH, temperature, and dissolved oxygen within the ranges chosen for evaluation had only a marginal impact on antibody expression in glycoengineered yeast but found that methanol feed rate had a substantial impact on productivity. Optimization of the methanol fed rate has been done by evaluating different methanol feeding regimes including maintaining a fixed methanol concentration (Damasceno et al., 2004), controlling dissolved oxygen concentration with methanol feed rate (Charoenrat et al., 2005), carbon limited feed strategies (Zhang et al., 2000) as well as mixed carbon source feeds (Ramon et al., 2007).

Several reports have indicated that medium composition can influence heterologous protein expression in yeast by affecting cell growth and viability, or altering the secretion of extracellular proteases. The addition of sorbitol or betaine to culture media to apply high osmotic stress was reported as a novel strategy to increase production of a heterologous enzyme several hundred fold in E. coli (Blackwell, J R and Horgan, R., 1991). Shi et al. reported that culturing P. pastoris in hypertonic media containing either 0.35 M potassium acetate, 0.35 M sodium chloride, or 1.0 M sorbitol during pre-induction biomass accumulation (ie., prior to induction) and then transferring to induction medium led to increased single-chain antibody (scFv) production. However, unlike the results reported for E. coli, the cultivation of Pichia in media including either betaine or spermine did not result in an additional increase in heterologous protein expression (Shi, X et al. 2003). Shi et al. further report that increasing the hypertonicity of Pichia induction medium led to rapid cell lysis and little scFv production.

One skilled in the art of fermentation will acknowledge that in order to maximize product yield it is critical to monitor and control several culture parameters throughout the fermentation process. For example, parameters such as temperature, dissolved oxygen levels, pH, agitation, aeration, nature of carbon source and feed-rates are typically monitored and controlled, in order to maximize expression levels. Partial pressure of carbon dioxide (pCO2), pH, and their associated osmolality in medium have been reported to influence tPA (Tissue Plasminogen Activator) production in CHO cell (Zanghi 1999). Compared to normal osmolality of medium at 310 mOsm/kg H₂O, hyper-osmolality over 500 mOsm/kg H₂O has been shown to improve cell viability and titer of foreign proteins such as INF-β, tPA, and antibodies in CHO cell culture (Kim 2000, Ryu 2000, Han 2009, Pacis 2011). Other studies report that the addition of an organic nitrogen source (e.g., a mixture of yeast extract and peptone) to a Pichia fed-batch culture system improved expression levels of mouse α-amylase (Choi and Park 2006). Therefore, because the composition of medium used for cultivation of host cells is known to effect the physiological phenotype, viability and production yields of host cells (e.g., production strains), media optimization has become an integral aspect of commercial scale bioprocess development.

Under carbon-limited fed-batch cultivation as a platform method, the osmolality of BSGY batch medium containing 4% glycerol as a carbon source is 1300±30 mOsm/kg H₂O and the osmolality continuously decreases as fermentation time increases. For example, the osmolality can decrease to 500±25 mOsm/kg H₂O at the end of glycerol fed-batch and 400±20 mOsm/kg H₂O at the end of induction phase (>48 h). The osmolality profile is highly dependent on medium formulation, growth rate of P. pastrois in each growth phase, and methanol consumption rate during the induction phase. As reported herein, the invention is based on the observation that compared to the wild type of P. pastoris, some glycol-engineered Pichia strains are less tolerant to the hypo-osmotic condition, e.g., 400±20 mOsm/kg H₂O that are characteristic of the methanol/induction phase.

The general principles of P. pastoris fermentation processes and the strategies for improving productivity still apply for glycoengineered Pichia strains. Using optimized process parameters and cultivation conditions defined using a design of experiments approach it has been reported that a glycoengineered production strain, which is capable of producing humanized glycoproteins with a terminal galactose, can be used for monoclonal antibody production on a commercial scale (Ye et al. Biotechnol. Prog. (2011). The resulting process yielded up to 1.6 g/L of monoclonal antibody and was scaled-up to the 1,200-L scale. The final product profile was observed to comprise more than 95% intact antibody possessing the desired complex N-glycan profile and acceptable quality attributes of protein aggregation and degradation. Optimized cultivation parameters included pH, temperature, methanol feed rate, biomass at induction and duration of induction phase. An earlier study reported the use of a different glycoengineered Pichia strain capable of producing more than 1 g/L of human IgG with greater than 90% homogeneous Man₅GlcNAc₂N-glycans across a range of fermentation conditions (Potgieter et al., J. Biotechnol. (2010)).

Jacobs et al reported the results of a study using a GlycoSwitch-Man₅ Pichia production strain to produce murine granulocyte-macrophage colony-stimulating factor (GM-CSF) as a test protein using different fed-batch fermentation strategies (Jacobs et al. Microbial Cell Factories 9:93 (2010)). The primary goal of the study was to determine the robustness of the strains in terms of N-glycan homogeneity and product yield when subjected to different feeding strategies (methanol-excess feed and methanol-limited feed). The results of the GM-CSF study revealed that growth rate does not significantly affect N-glycan homogeneity, however there was a clear relationship between product yield and specific growth rate. It was also reported that limiting the growth rate during the induction phase to approximately 25% of the maximum specific growth rate resulted in maximum GM-CSF yield (expressed as yield per unit volume). This is consistent with the theory that specific productivity of cells can be optimized by controlling the specific growth rate of the host cell population. Considered together the results indicate that conversion of the P. pastoris N-glycosylation pathway to produce proteins with humanized glycosylation profiles does not significantly alter the yeast's physiology and that glycoengineered strains are fully capable of producing diverse mammalian glycoproteins on a commercial scale.

Several sugars and sugar alcohols are known to be osmolytes for yeast cells. Sugar sources such as sucrose, glycerol, sorbitol, and arabitol are well known as compatible solutes in P. pastoris to modulate osmotic pressure of the cell and to enhance protein stability (Arakawa 2007). Glycine betaine is another component used to protect bacteria (e.g., Escherichia coli and Gluconacetobacter diazotrophicus), as well as eukaryotic cells such as yeast, and CHO cells at hyper-osmotic condition (Boniolo 2009, Kiewietdejonge 2006, Kim 2000, Ryu 2000). Trehalose is known to be accumulated through de novo biosynthesis by bacteria in response to abiotic stresses (Dominguez-Ferreras 2009).

Generally speaking osmolytes are compounds present in solution within a cell or its surrounding fluid that affect osmosis. Osmolytes play a role in maintaining cell volume and fluid balance. Glycerol, arabitol, sorbitol, and trehalose have been utilized for modulating cellular osmotic pressure under osmotic stress conditions (Hohmann 2002; Dragosits 2010; Gorka-Niec 2010; Van der Heide 2000). As used herein the term “osmoprotectant” refers to compatible solutes or small molecules that act as an osmolyte.

In their natural habitats, yeast cells are often exposed to drastic changes in osmolarity. In response to changes in environmental osmolarity, yeast cells execute an adaptive process to adjust their intracellular solute concentrations in order to maintain a constant turgor pressure and ensure cellular activity (Kayingo et al. 2001). Generally speaking, biotechnological production processes aim at high cell and product concentrations of nutrient salts and carbon sources which can result in transient high osmolarity. During a complete cultivation/fermentation protocol production processes can expose yeast host cells to changing culture conditions from an initial physiologic osmolarity to conditions that can range between hypo-osmotic to hyper-osmotic. The range of physiological osmolality for a Pichia fermentation culture is considered to be about 450 to about 1900 mOsm/kg H₂O. Pichia strains in the study were not able to grow at normal growth rate at hyper-osmotic condition higher than 1900 mOsm/kg H₂O.

Generally the osmolality of medium decreases as the nutrient level decreases due to cellular consumption by the cells. We observed that the average cell size of Pichia tended to increase as fermentation time increased, especially during induction phases under regular feeding conditions including carbon-limited and oxygen-limited conditions. We assumed that the cells were exposed to nutrient depleted (hypo-osmotic) condition and were swollen by increased the influx of water into the cell. Under this condition, the yeast cell is prone to lysis. Changes in the structure of cell wall or membrane of highly glycol-engineered Pichia strains make these strains more susceptible to lysis relative to the susceptibility of wild type production strains exposed to the same media and cultivation conditions. The volumetric productivity of protein (P, g/L) is proportional to the induction time if the specific productivity (Qp, g/g/h) is not changed in different culture conditions. As used herein the term “volumetric productivity” means the amount of target protein per unit volume of culture (g/L).

The addition of nutrient components metabolized by Pichia cells is know to transiently increase the osmolality of culture medium but this has not been observed to confer a measurable effect on cell viability or increased volumetric productivity. The transient effect is likely attributed to the consumption of fermentable substrates, co-facors or nutrients by the culture.

The most pronounced cellular response to osmotic stress is the production and regulation of osmoprotective solutes (i.e., osmoyltes) such as glycerol, arabitol, or mannitol. Exposure of production strains to hypo-osmotic shock results in a rapid inflow of water and cell swelling, which increases the turgor pressure and could result in cell lysis. To circumvent cellular lysis, the cell utilizes a regulatory-volume process to adjust its intracellular solute levels. It is known that the yeast responses the osmotic stress via pathways of Cell Wall Integrity (CWI) under hypotonic environment and High Osmolarity Glycerol (HOG) under hypertonic condition.

Mammalian cells also respond to hypo-osmostic shock by rapidly releasing osmolytes including both organic and inorganic solutes into the culture media. The prior art includes reports provide evidence that the exposure to osmotic stress can have a beneficial effect on recombinant protein production in bacteria, yeast and mammalian host cells (Dragosits et al 2010). For example, the intentional exposure of hybridoma cells to hyperosmotic pressure, by the addition of sodium chloride, to the culture media was recognized as an economical way to increase the specific antibody productivity in hybridoma cell cultures (Ryu et al. 2000). However, because cell growth was found to be suppressed at the elevated osmolality, the use of hyperosmolar medium in batch culture did not significantly increase antibody yield. It was subsequently discovered that inclusion of the osmoprotective compound glycine betaine, in the hyperosmolar medium could improve cell growth to a level where up to a twofold increase in maximum antibody concentration was achieved relative to a control culture in media with physiological osmolality (Oyass et al., 1994). It was appreciated that if a similar pattern of response could be obtained in hyperosmotic recombinant Chinese Hamster Ovary (CHO) cell cultures, the use of glycine betaine as an osmoprotectant would have a great impact on the economical production of heterologous proteins in CHO cells. However, the effect of glycine betaine on the production of recombinant heterologous proteins in CHO cell lines was found to be cell line specific (Ryu et al. 2000). With regard to the effects of osmotic stress (i.e., increasing osmolarity) on yeast host cells the evidence has been largely anecdotal, and nonexistent for glycoengineered yeast strains.

The present invention is based on the discovery that particular sugars and sugar alcohols such as maltose, sorbose, ribose, maltitol, myo-inositol, mellibiose, and quinic acids can be used as osmoprotectants to increase and maintain the osmolality of the cultivation medium in order to protect Pichia host cells from lysis under hypo-osmotic condition, which results from depletion or limitation of substrates in the medium during methanol induction phase in high-cell-density cultures. By using an osmoprotectant of the invention to increase and maintain the osmolality of the culture media during the induction phase, it is expected that longer culture times (eg., longer induction times) will result in increased volumetric productivity of the culture.

Osmolality of the medium (Φ(t), mOsm/kg) is determined by summation of osmolality of individual component in the medium at culture time (t), as shown in Eq (1).

$$Φ ( t ) = {\sum }_{i}^{ } {Φ}_{i} ( t ) \qquad ( 1 )$$

Here, Φᵢ(t) is the osmolality of i-th component in the medium as follows,

Φᵢ(t)=φᵢ·nᵢ·Cᵢ(t) (2)

Where, for i-th component, Φᵢ is the osmotic coefficient (mOsm/kg/mol), nᵢ is the number of dissociated molecules in solution and Cᵢ(t) is the concentration (mol) at culture time (t) in Eq (2).

Osmolality is proportional to the concentration of nutrients in the culture medium as defined in Eqs (1) and (2). Osmolality is the concentration of a solution in terms of osmoles per kilogram of solvent. Osmolarity is the concentration of a solution in terms of solutes per liter of solution.

### ABBREVIATIONS

Φ(t) Overall Osmolality (mOsm/kg) at culture time (t)

Φᵢ(t) Osmolality (mOsm/kg) of i-th component of medium at culture time (t)

φᵢ Osmolality coefficient (mOsm/kg/mol) of i-th component of medium

nᵢ The number of dissociated molecules from i-th component in medium

Cᵢ(t) The concentration (mol) of i-th component in medium at culture time (t)

Qₛ Specific consumption rate of substrate (h⁻¹)

μ_{B} Specific growth rate of cell in batch phase (h⁻¹)

μ_{G} Specific growth rate of cell in glycerol fed-batch phase (h⁻¹)

μ_{M} Specific growth rate of cell in methanol fed-batch phase (h⁻¹)

Typically, when P. pastoris grows in BSGY (BSGY (g/L)=yeast extract (10)+soytone (20)+KH2PO4 (2.3)+K2HPO4 (11.9)+Yeast Nitrogen Base without amino acids (13.4)+D-Biotin (0.008)+Sorbitol (18.2)+Glycerol (40)) medium, the osmolality of medium is gradually reduced during the culture due to uptake of substrates as nutrient components. If the substrate is fermentable, it is transported into the cell and actively metabolized for cell growth or maintaining cell viability. If the substrate is non-fermentable, it may or may not be transported into the cell, but it is never metabolized by the cell. Therefore, the osmolality of the culture media changes as a result of the addition and consumption of a fermentable substrate, but it does not change as a consequence of the addition of a non-fermentable substrate.

The consumption rate of fermentable-substrates (Qₛ, g/g/h depends on the growth rate. Typically Qₛ is highest in initial batch growth phase because cells can grow at the maximal growth rate (μ_{B}>0.1 h⁻¹). The Qₛ becomes lower in glycerol fed-batch period due to lower cell growth rate (e.g., μ_{G}=0.08 h⁻¹) and even much lower in methanol induction phase (e.g., μ_{M}=0.008 h⁻¹). The osmolality during the cultivation was modulated by using different combinations of non-fermentable sugars or sugar alcohols which were being evaluated as osmoprotectants in combination with other nutrient components, including but not limited to sorbitol, and media supplements including amino acid, vitamin, trace metal and basal salt mixes.

The results presented herein illustrate that glycoengineered Pichia host strains cultured under relevant bioprocess conditions in the presence of certain osmoprotectants (i.e., particular non-fermentable sugars and sugar alcohols), were found to exhibit improved viability, stability, and protein production. More specifically, as shown herein, maltose (a non-fermentable sugar) and maltitol (a non-fermentable sugar alcohol) significantly improved the robustness of Pichia production strains during the methanol induction phase of cultivation. As used herein the term “robustness” refers to improved cellular fitness as determined by improved viability, cellular integrity, and product yield relative to the level of viability, cellular integrity or product yield observed in a culture of the same production strain in the absence of an osmoprotectant of the invention.

Generally speaking, inclusion of an osmoprotectant in the methanol fed-batch media improves cell viability. For example, as shown herein, a cellular viability of 70% for a gycoengineered Pichia strain producing insulin, has been improved to a level of 95% by supplementing BSGY with an osmoprotectant of the invention. The improved cellular robustness allowed the induction phase of the insulin producing production strain to extended to 83 hours, which is 24 hours longer than average induction stages for cultures using this stain in the absence of an osmoprotectant of the invention. The induction phase of a different production strain, producing human Fc, is demonstrated to be extended by 3 days (e.g., 72 hours). Using the methods of the invention, it is likely that the methanol induction phase of any given production strain can be extended by more than about 1, 2, 3, or 4 days.

As shown herein, the improved viability and extended methanol induction production phase can result in improved yields (e.g., volumentric productivity) by more than 1.2-fold (e.g., 20%). It is likely that the fold of improvement will depend upon the production strain and the nature of the protein that is being produced. In practice, it is likely that the disclosed osmoprotectants and improved production processes can be used to improve the volumetric productivity of Pichia strains by 20%, 25%, 30%, 35%, 40%, 45%, 50%, 55%, 60%, 65%, 70%, 75%, 80%, 85%, 90%, 95% or more than 100%. As shown herein, use of an osmprotectant of the invention in accordance with the improved production process increased product yield 1.2-fold for yGLY13979 producing an anti-Her2 antibody or approximately 2-fold for yGLY21058) producing insulin.

As used herein a sugar alcohol (also known as a polyol or poly or a polyalcohol) refers to a hydrogenated form of a carbohydrate whose carbonyl group has been reduced to a primary or secondary hydroxyl group. Sugar alcohols have the general formula H(HCHO)ₙ₊₁H while sugars have the formula H(HCHO)ₙHCO. Nonfermentable sugars and sugar alcohols such as maltose, sorbose, ribose, maltitol, myo-inositol, mellibiose, and quinic acids are newly identified as osmoprotectants useful for increasing and maintaining osmolality to protect Pichia cells, and in particular engineered Pichia cells from lysis under hypo-osmotic condition, which results from depletion or limitation of substrates in the medium during methanol induction phase in high-cell-density cultures. In particular, maltose and maltitol, a non-fermentable sugar and sugar alcohol, respectively, significantly improved robustness of glycoengineered Pichia cells.

Host Cells The present invention encompasses any isolated Pichia sp. host cell (e.g., such as Pichia pastoris) comprising various modified constructs, including host cells comprising a promoter e.g., operably linked to a polynucleotide encoding a heterologous polypeptide (e.g., a reporter or immunoglobulin heavy and/or light chain) as well as methods of use thereof, e.g., methods for expressing the heterologous polypeptide in the host cell. Host cells of the present invention, may be also genetically engineered so as to express particular glycosylation patterns on polypeptides that are expressed in such cells. Host cells of the present invention are discussed in detail herein. Any engineered Pichia host cell cultured under any of the described conditions forms part of the present invention. In an embodiment of the invention, the host cell is selected from the group consisting of any Pichia cell, such as Pichia pastoris, Pichia angusta (Hansenula polymorpha), Pichia flnlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia.

As used herein, the terms “N-glycan” and “glycoform” are used interchangeably and refer to an N-linked oligosaccharide, e.g., one that is attached by an asparagine-N-acetylglucosamine linkage to an asparagine residue of a polypeptide. N-linked glycoproteins contain an N-acetylglucosamine residue linked to the amide nitrogen of an asparagine residue in the protein. Predominant sugars found on glycoproteins are glucose, galactose, mannose, fucose, N-acetylgalactosamine (GalNAc), N-acetylglucosamine (GlcNAc) and sialic acid (e.g., N-acetyl-neuraminic acid (NANA)).

N-glycans have a common pentasaccharide core of Man₃GlcNAc₂ (“Man” refers to mannose; “Glc” refers to glucose; and “NAc” refers to N-acetyl; GlcNAc refers to N-acetylglucosamine). N-glycans differ with respect to the number of branches (antennae) comprising peripheral sugars (e.g., GlcNAc, galactose, fucose and sialic acid) that are added to the Man₃GlcNAc₂ (“Man₃”) core structure which is also referred to as the “trimannose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). Complex N-glycans may also have multiple antennae on the “trimannose core,” often referred to as “multiple antennary glycans.” A “hybrid” N-glycan has at least one GlcNAc on the terminal of the 1,3 mannose arm of the trimannose core and zero or more mannoses on the 1,6 mannose arm of the trimannose core. The various N-glycans are also referred to as “glycoforms.” “PNGase”, or “glycanase” or “glucosidase” refer to peptide N-glycosidase F (EC 3.2.2.18).

In an embodiment of the invention, a Pichia host cell (e.g., Pichia pastoris) is cultured under the conditions (pH 6.5, 24° C. in complex medium (e.g., BSGY)), and additionally the Pichia host cell has been genetically engineered to include a nucleic acid that encodes an alpha-1,2-mannosidase that has a signal peptide that directs it for secretion. For example, in an embodiment of the invention, the host cell is engineered to express an exogenous alpha-1,2-mannosidase enzyme having an optimal pH between 5.1 and 8.0, preferably between 5.9 and 7.5. In an embodiment of the invention, the exogenous enzyme is targeted to the endoplasmic reticulum or Golgi apparatus of the host cell, where it trims N-glycans such as Man₈GlcNAc₂ to yield ManₛGlcNAc₂. See U.S. Pat. No. 7,029,872.

In an embodiment of the invention, Pichia host cells (e.g., Pichia pastoris) cultured under conditions of the present invention, are also genetically engineered to eliminate glycoproteins having alpha-mannosidase-resistant N-glycans by deleting or disrupting one or more of the beta-mannosyltransferasegenes (e.g., BMT1, BMT2, BMT3, and BMT4)(See, U.S. Pat. No. 7,465,577) or abrogating translation of RNAs encoding one or more of the beta-mannosyltransferasesusinginterfering RNA, antisense RNA, or the like. The scope of the present invention includes such cultured engineered Pichia host cells (e.g., Pichia pastoris) comprising an expression cassette (e.g., a promoter operably linked to a heterologous polynucleotide encoding a heterologous polypeptide).

Engineered host cells (e.g., Pichia pastoris) cultured under conditions of the present invention also include those that are genetically engineered to eliminate glycoproteins having phosphomannose residues, e.g., by deleting or disrupting one or both of the phosphomannosyl transferase genes PNO1 and MNN4B (See for example, U.S. Pat. Nos. 7,198,921 and 7,259,007), which can include deleting or disrupting the MNN4A gene or abrogating translation of RNAs encoding one or more of the phosphomannosyltransferases using interfering RNA, antisense RNA, or the like. In an embodiment of the invention, an engineered Pichia host cell has been genetically modified to produce glycoproteins that have predominantly an N-glycan selected from the group consisting of complex N-glycans, hybrid N-glycans, and high mannose N-glycans wherein complex N-glycans are, in an embodiment of the invention, selected from the group consisting of Man₃GlcNAc₂, GlcNAC₍₁₋₄₎Man₃GlcNAc₂, NANA₍₁₋₄₎GlcNAc₍₁₋₄₎Man₃GlcNAc₂, and NANA₍₁₋₄₎Gal₍₁₋₄₎Man₃GlcNAc₂; hybrid N-glycans are, in an embodiment of the invention, selected from the group consisting of Man₅GlcNAc₂, GlcNAcMan₅GlcNAc₂, GalGlcNAcMan₅GlcNAc₂, and NANAGalGlcNAcMan₅GlcNAc₂; and high mannose N-glycans are, in an embodiment of the invention, selected from the group consisting of Man₆GlcNAc₂, Man₇GlcNAc₂, Mang₈lcNAc₂, and Man₉GlcNAc₂. The scope of the present invention includes such engineered Pichia host cells (e.g., Pichia pastoris) comprising a modified, truncated, or deleted form of the XRN1 gene.

Additional embodiments of the present invention include engineered Pichia host cells (e.g., Pichia pastoris) cultured under conditions of the present invention that are genetically engineered to include a nucleic acid that encodes the Leishmania sp. single-subunit oligosaccharyltransferase STT3A protein, STT3B protein, STT3C protein, STT3D protein, or combinations thereof such as those described in WO2011/06389. Additionally, engineered host cells (e.g., Pichia pastoris) cultured under conditions of the present invention also include those that are genetically engineered to eliminate nucleic acids encoding Dolichol-P-Man dependent alpha(1-3) mannosyltransferase, e.g., Alg3, such as described in US Patent Publication No. US2005/0170452. The scope of the present invention includes any such engineered Pichia host cells (e.g., Pichia pastoris) further comprising a modified, truncated, deleted form of the XRN1 gene.

As used herein, the term “essentially free of” as it relates to lack of a particular sugar residue, such as fucose, or galactose or the like, on a glycoprotein, is used to indicate that the glycoprotein composition is substantially devoid of N-glycans which contain such residues. Expressed in terms of purity, essentially free means that the amount of N-glycan structures containing such sugar residues does not exceed 10%, and preferably is below 5%, more preferably below 1%, most preferably below 0.5%, wherein the percentages are by weight or by mole percent.

As used herein, a glycoprotein composition “lacks” or “is lacking” a particular sugar residue, such as fucose or galactose, when no detectable amount of such sugar residue is present on the N-glycan structures. For example, in an embodiment of the present invention, glycoprotein compositions produced by host cells of the invention will “lack fucose,” because the cells do not have the enzymes needed to produce fucosylated N-glycan structures. Thus, the term “essentially free of fucose” encompasses the term “lacking fucose.” However, a composition may be “essentially free of fucose” even if the composition at one time contained fucosylated N-glycan structures or contains limited, but detectable amounts of fucosylated N-glycan structures as described above.

As used herein, the term methanol-induction refers to increasing expression of a polynucleotide (e.g., a heterologous polynucleotide) operably linked to a methanol-inducible promoter in a host cell of the present invention by exposing the host cells to methanol.

As used herein, term methanol-repression refers to decreasing expression of a polynucleotide (e.g., a heterologous polynucleotide) operably linked to a methanol-repressible promoter in a host cell of the present invention by exposing the host cells to methanol.

As used herein the term “Non-fermentable sugars and sugar alcohols” is defined to encompass both “strict non-fermentable” (e.g. including, but not limited to D-maltose, maltitol, and D-gluconic acid), and “non-active fermentable” (e.g., including but not limited to ones like D-sorbose, D-ribose, myo-ibositol, and L-melibiose) sugars and sugar alcohols.

### Molecular Biology

In accordance with the present invention there may be employed conventional molecular biology, microbiology, and recombinant DNA techniques within the skill of the art. Unless otherwise defined herein, scientific and technical terms used in connection with the present invention shall have the meanings that are commonly understood by those of ordinary skill in the art. Further, unless otherwise required by context, singular terms shall include the plural and plural terms shall include the singular. Generally, nomenclatures used in connection with, and techniques of biochemistry, enzymology, molecular and cellular biology, microbiology, genetics and protein and nucleic acid chemistry and hybridization described herein are those well known and commonly used in the art.

The methods and techniques of the present invention are generally performed according to conventional methods well known in the art and as described in various general and more specific references that are cited and discussed throughout the present specification unless otherwise indicated. See, e.g., James M. Cregg (Editor), Pichia Protocols (Methods in Molecular Biology), Humana Press (2010), Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999), Animal Cell Culture (R. I. Freshney, ed. (1986)); Immobilized Cells And Enzymes (IRL Press, (1986)); B. Perbal, A Practical Guide To Molecular Cloning (1984);

A “polynucleotide”, “nucleic acid” includes DNA and RNA in single stranded form, double-stranded form or otherwise.

A “polynucleotide sequence” or “nucleotide sequence” is a series of nucleotide bases (also called “nucleotides”) in a nucleic acid, such as DNA or RNA, and means a series of two or more nucleotides. Any polynucleotide comprising a nucleotide sequence set forth herein (e.g., promoters of the present invention) forms part of the present invention.

A “coding sequence” or a sequence “encoding” an expression product, such as an RNA or polypeptide is a nucleotide sequence (e.g., heterologous polynucleotide) that, when expressed, results in production of the product (e.g., a heterologous polypeptide such as an immunoglobulin heavy chain and/or light chain).

As used herein, the term “oligonucleotide” refers to a nucleic acid, generally of no more than about 100 nucleotides (e.g., 30, 40, 50, 60, 70, 80, or 90), that may be hybridizable to a polynucleotide molecule. Oligonucleotides can be labeled, e.g., by incorporation of ³²P-nucleotides, ³H-nucleotides, ¹⁴C-nucleotides, ³⁵S-nucleotides or nucleotides to which a label, such as biotin, has been covalently conjugated.

A “protein”, “peptide” or “polypeptide” (e.g., a heterologous polypeptide such as an immunoglobulin heavy chain and/or light chain) includes a contiguous string of two or more amino acids.

A “protein sequence”, “peptide sequence” or “polypeptide sequence” or “amino acid sequence” refers to a series of two or more amino acids in a protein, peptide or polypeptide.

The term “isolated polynucleotide” or “isolated polypeptide” includes a polynucleotide or polypeptide, respectively, which is partially or fully separated from other components that are normally found in cells or in recombinant DNA expression systems or any other contaminant. These components include, but are not limited to, cell membranes, cell walls, ribosomes, polymerases, serum components and extraneous genomic sequences. The scope of the present invention includes the isolated polynucleotides set forth herein, e.g., the promoters set forth herein; and methods related thereto, e.g., as discussed herein.

An isolated polynucleotide or polypeptide will, preferably, be an essentially homogeneous composition of molecules but may contain some heterogeneity.

“Amplification” of DNA as used includes the use of polymerase chain reaction (PCR) to increase the concentration of a particular DNA sequence within a mixture of DNA sequences. For a description of PCR see Saiki, et al., Science (1988) 239:487.

In general, a “promoter” or “promoter sequence” is a DNA regulatory region capable of binding an RNA polymerase in a cell (e.g., directly or through other promoter-bound proteins or substances) and initiating transcription of a coding sequence to which it operably links.

A coding sequence (e.g., of a heterologous polynucleotide, e.g., reporter gene or immunoglobulin heavy and/or light chain) is “operably linked to”, “under the control of”, “functionally associated with” or “operably associated with” a transcriptional and translational control sequence (e.g., a promoter of the present invention) when the sequence directs RNA polymerase mediated transcription of the coding sequence into RNA, preferably mRNA, which then may be RNA spliced (if it contains introns) and, optionally, translated into a protein encoded by the coding sequence.

The present invention includes vectors or cassettes which comprise various modified constructs, including promoters optionally operably linked to a heterologous polynucleotide. The term “vector” includes a vehicle (e.g., a plasmid) by which a DNA or RNA sequence can be introduced into a host cell, so as to transform the host and, optionally, promote expression and/or replication of the introduced sequence. Suitable vectors for use herein include plasmids, integratable DNA fragments, and other vehicles that may facilitate introduction of the nucleic acids into the genome of a host cell (e.g., Pichia pastoris). Plasmids are the most commonly used form of vector but all other forms of vectors which serve a similar function and which are, or become, known in the art are suitable for use herein. See, e.g., Pouwels, et al., Cloning Vectors: A Laboratory Manual, 1985 and Supplements, Elsevier, N.Y., and Rodriguez et al. (eds.), Vectors: A Survey of Molecular Cloning Vectors and Their Uses, 1988, Buttersworth, Boston, Mass.

A polynucleotide (e.g., a heterologous polynucleotide, e.g., encoding an immunoglobulin heavy chain and/or light chain), operably linked to a promoter, may be expressed in an expression system. The term “expression system” means a host cell and compatible vector which, under suitable conditions, can express a protein or nucleic acid which is carried by the vector and introduced to the host cell. Common expression systems include fungal host cells (e.g., Pichia pastoris) and plasmid vectors, insect host cells and Baculovirus vectors, and mammalian host cells and vectors.

The following references regarding the BLAST algorithm are herein incorporated by reference: BLAST ALGORITHMS: Altschul, S. F., et al., J. Mol. Biol. (1990) 215:403-410; Gish, W., et al., Nature Genet. (1993) 3:266-272; Madden, T. L., et al., Meth. Enzymol. (1996) 266:131-141; Altschul, S. F., et al., Nucleic Acids Res. (1997) 25:3389-3402; Zhang, J., et al., Genome Res. (1997) 7:649-656; Wootton, J. C., et al., Comput. Chem. (1993) 17:149-163; Hancock, J. M., et al., Comput. Appl. Biosci. (1994) 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found, Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found, Washington, D.C.; Altschul, S. F., J. Mol. Biol. (1991) 219:555-565; States, D. J., et al., Methods (1991) 3:66-70; Henikoff, S., et al., Proc. Natl. Acad. Sci. USA (1992)89:10915-10919; Altschul, S. F., et al., J. Mol. Evol. (1993) 36:290-300; ALIGNMENT STATISTICS: Karlin, S., et al., Proc. Natl. Acad. Sci. USA (1990) 87:2264-2268; Karlin, S., et al., Proc. Natl. Acad. Sci. USA (1993) 90:5873-5877; Dembo, A., et al., Ann. Prob. (1994) 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

The present invention is not to be limited in scope by the specific embodiments described herein; the embodiments specifically set forth herein are not necessarily intended to be exhaustive. Indeed, various modifications of the invention in addition to those described herein will become apparent to those skilled in the art from the foregoing description and the accompanying figures. Such modifications are intended to fall within the scope of the appended claims.

### EXAMPLES

The following examples are intended to exemplify the present invention and not to be a limitation thereof. The methods and compositions disclosed below fall within the scope of the present invention.

### Experimental Methods

Fed-batch fermentations, IgG purifications, N-glycan characterizations, as well as all other analytical assays, were performed as previously described (Barnard et al. 2010; Jiang et al. 2011; Potgieter et al. 2009; Winston F 2008). Generally speaking, the Pichia host cells (e.g., Pichia pastoris) used in the following examples have been genetically engineered to include a nucleic acid that encodes an alpha-1,2-mannosidase and utilize signal peptides to direct secretion of the heterologous protein product. Typical culture conditions are pH 6.5, at 24° C. in complex medium (e.g., BSGY)) with and without supplements.

BSGY medium comprises (g/L)=yeast extract (10)+soytone (20)+KH2PO4 (2.3)+K2HPO4 (11.9)+Yeast Nitrogen Base without amino acids (13.4)+D-Biotin (0.008)+Sorbitol (18.2)+Glycerol (40).

### Production Strains

Glycoengineered Pichia production strains used in the examples include (yGLY21058, GS6.0) producing glycosylated insulin precursor, (yGLY27893, GS6.0) producing human Fc fragment and (yGLY13979, GS5.0) producing anti-Her2 monoclonal antibody.

### Example 1

### Osmolality of Medium Components

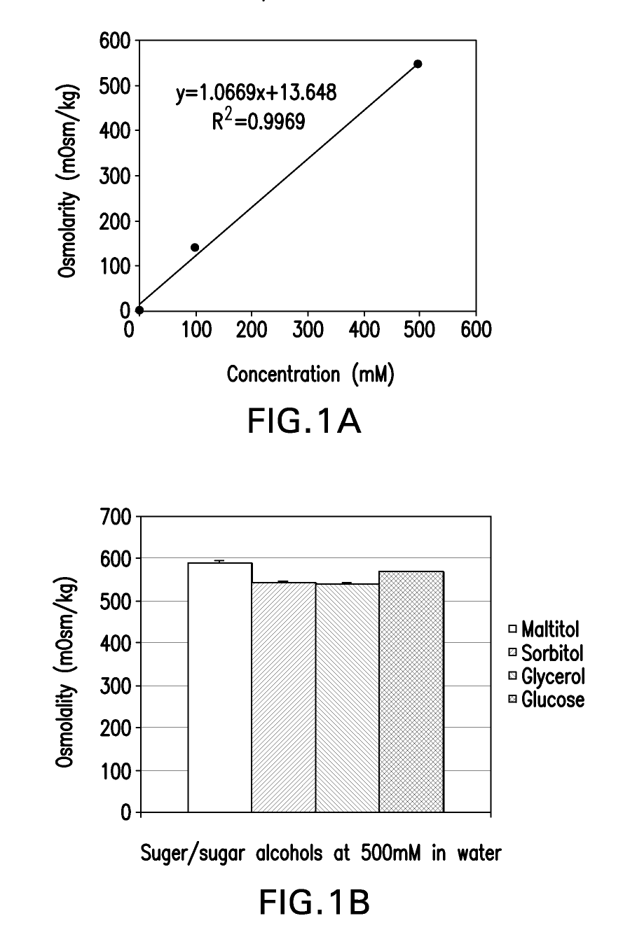

The sugar and sugar alcohol solutions used as osmoprotectant supplements were prepared at 500 mM in water and their osmolalities were measured using Osmometer (Multi-somotte, Model 2430, Precision Systems Inc, MA, USA). The osmolality of solution was directly proportional to the concentration of component in solution. For instance there was a linear relationship between sorbitol concentration (mM) and osmolality of the solution (mOsm/kg) as shown in FIG. 1A. The osmolalities of maltitol, sorbitol, glycerol, and glucose at 500 mM in water were 590±4, 544±3, 541±3, and 568±0 mOsm/kg respectively (FIG. 1B). The osmolaties of sorbose, mellibiose, ribose, quinic acid and myo-inositol at 500 Mm in water were (480±5), (500±5), (470±5), (480±5) AND (510±10), respectively.

### Example 2

### Fermentability of Sugars and Sugar Alcohols for P. pastoris on Agar Plates and in Batch Medium

The 1 mL of RCB (Research Cell Bank in 20% of glycerol) for P. pastrois strain yGLY21058 producing glycosylated insulin precursor (GIP) was inoculated in 200 mL of Seed medium (4% glycerol, 1% yeast extract, 2% soytone, 1.34% YNB without amino acids, 0.23% K₂HPO₄, 1.19% KH₂PO₄, 8 μg/L biotin) and cultivated for 48 h at 24° C. The cell pellet was harvested by centrifugation at 4,000 rpm for 10 min and re-suspended with PBS buffer (pH 7.4) to wash the cell twice. The 0.1 mL of cell suspension with wash buffer was transferred and spread onto the minimal agar plate containing each sugar or sugar alcohol (1% w/v), which is listed in Table 1.

The plates were incubated for 6 days at 30° C. and were observed for cell growth to determine the fermentability of each sugar or sugar alcohol. YPD (1% yeast extract, 2% yeast peptone, and 1% glucose) agar plate was used for the positive control and a minimal agar plate without carbon sources (M) was used for negative control. Growth of Pichia pastoris on M agar plate containing single carbon source was categorized as no (−), slow (+), moderate (++), and fast (+++) colony formation for 6 days in Table 1. The results indicate that the sugars D-arabinose and D-maltose, and the sugar alcohols L-arabitol, xylitol, D-ribitol, D-glucono-1,5-lactone, D-quinic acid, and maltitol represent strict non-fermentable substrates. D-xylose, D-ribose, L-sorbose, L-melibiose, inulin, myo-inositol, and D-gluconate showed very slow colony formation on the plate, which was categorized as “non-active fermentable substrates”.

TABLE 1
Fermentability of sugars or sugar alcohols on M agar plate

| Type | Name | Growth on M agar |
|---|---|---|
| Sugar | D-Arabinose | − |
| Sugar | D-Xylose | + |
| Sugar | D-Ribose | + |
| Sugar | D-Glucose | +++ |
| Sugar | L-Sorbose | + |
| Sugar | α,α-Trehalose | ++ |
| Sugar | D-Melibiose | + |
| Sugar | D-Maltose | − |
| Sugar | D-Raffinose | ++ |
| Sugar | Inulin | + |
| Sugar alcohol | Glycerol | +++ |
| Sugar alcohol | L-Arabitol | − |
| Sugar alcohol | Xylitol | − |
| Sugar alcohol | D-Ribitol | − |
| Sugar alcohol | D-Glucono-1,5- | − |
| | lactone | |
| Sugar alcohol | myo-Inositol | + |
| Sugar alcohol | D-Sorbitol | +++ |
| Sugar alcohol | D-Quinic acid | − |
| Sugar alcohol | D-Gluconate | + |
| Sugar alcohol | Maltitol | − |

### Example 3

### Screening of Sugars and Sugar Alcohols as Osmoprotectants for P. pastrois in Fed-Batch Cultivation

The P. pastoris YGLY21058 strain was cultivated in 1 L glass bioreactors (DASGIP, Germany). For fermentation in 1 L bioreactor, a vial (1 mL) of RCB (Research Cell Bank) was inoculated into 200 mL of Seed medium in 1 L-baffled flask. The culture incubated at 24° C., while shaking on an orbital shaker at 180 rpm for 48±4 h. The bioreactor was inoculated with a 10% volumetric ratio of seed to initial BSGY medium. Cultivation conditions were following: temperature set at 24±0.5° C., pH controlled at 6.5±0.1 with 30% ammonium hydroxide, dissolved oxygen was maintained at 20% of saturation by cascading agitation rate on the addition of pure oxygen to the fixed airflow rate of 0.7 vvm. After depletion of the initial glycerol (4%), a 50% glycerol solution containing 12.5 mL/L of PTM1 salts (6.5 g FeSO₄.7H₂O, 2.0 g ZnCl₂, 0.6 g CuSO₄.5H₂O, 3.0 g MnSO₄.7H₂O, 0.5 g CoCl₂.6H₂O, 0.2 g NaMoO₄.2H₂O, 0.2 g biotin, 80 mg NaI, 20 mg H₃BO₄ per L) was fed constantly at an initial rate of 0.08 h⁻¹ for 8 h. Induction was initiated after a 30 min starvation phase when methanol was fed. Methanol was fed constantly starting at 1.33 g/L/h under methanol limited condition. Agitation speed was changed from cascade mode with agitation speed and pure oxygen.

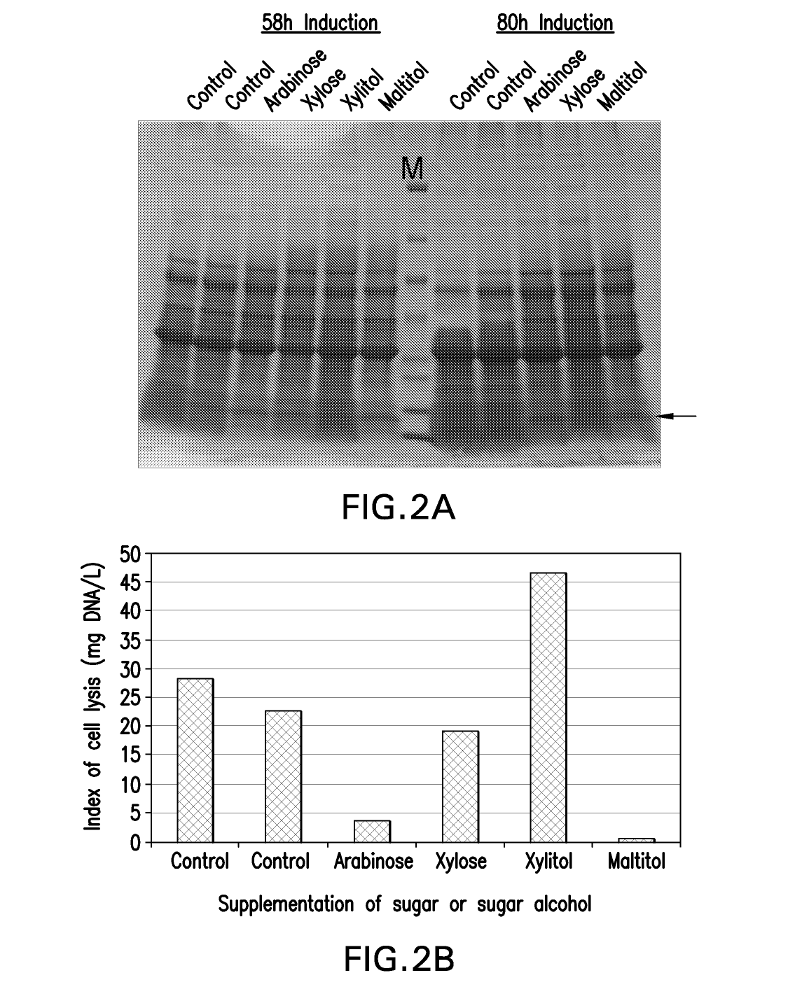

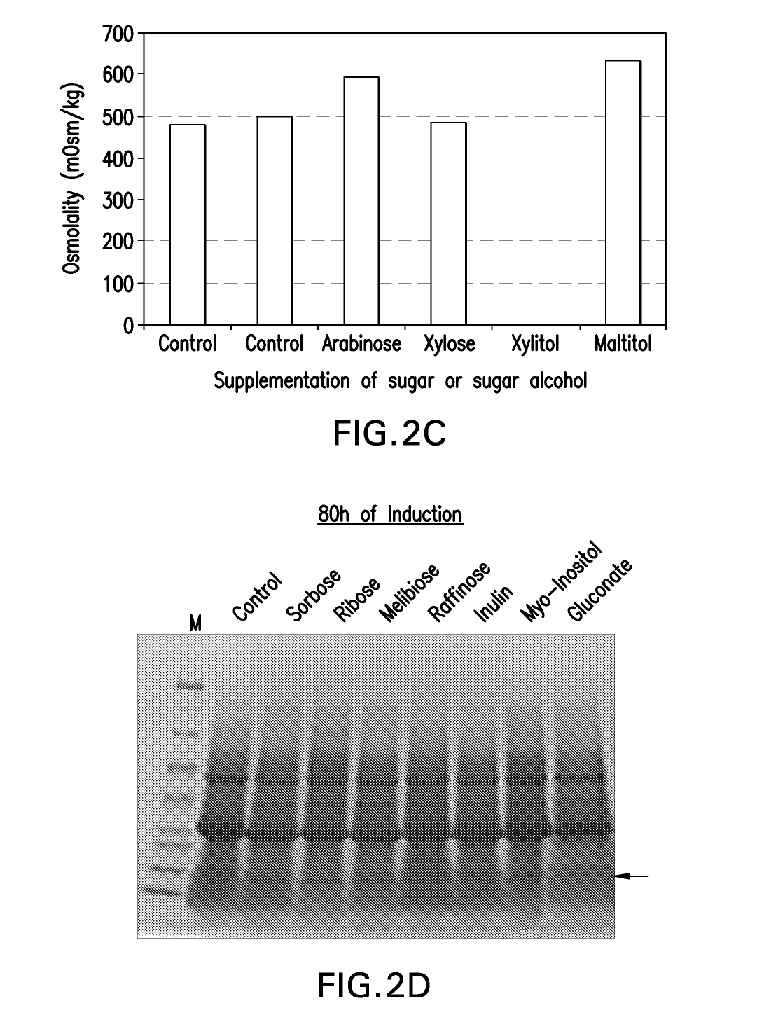

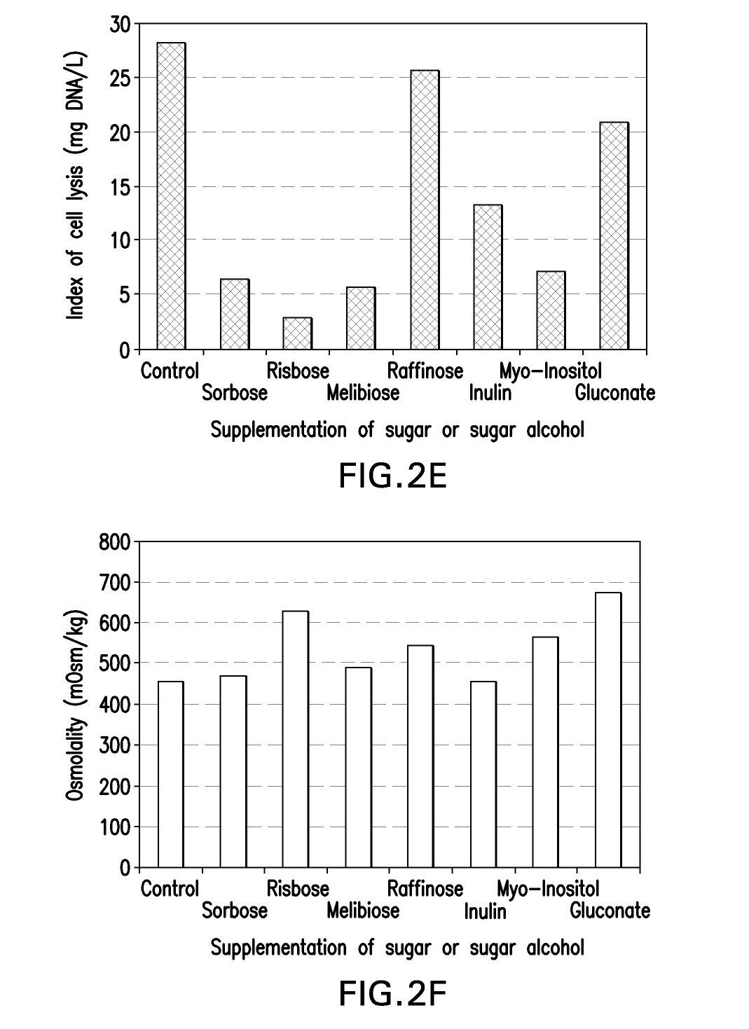

For comparisons, each sugar or sugar alcohol was added at 50 g/L in initial batch medium of BSGY. In case of control without sugar supplementation (BSGY medium only), glycosylated insulin precursor (GIP) was detectable at 58 h of induction, but not detected at 80 h of induction due to most likely proteolytic degradation caused by heavy cell lysis (Lanes 1 and 2 of FIG. 2 A). Similar results were observed in addition of xylose, xylitol, raffinose, and inulin in initial batch medium (FIGS. 2 A and D; as well as B and E). In contrast, intact GIP molecule was maintained in presence of arabinose, maltitol, sorbose, ribose, melibiose, and myo-inositol after 80 h-induction, which was strongly related with low indexes of cell lysis.

Significantly, the osmolality was maintained at higher levels (>470 mOsm/kg H₂O) in presence of these sugars or sugar alcohols (FIGS. 2 C and F). The index of cell lysis in these cases was less than 8 mg DNA/L (mg/L of double stranded DNA fragments released into the culture medium). The osmolality without sugar supplementation in this experiment was 420±20 at 58 h of induction before cell lysis and was slightly increased to 450±25 mOsm/kg H₂O at 80 h of induction with heavy cell lysis. The slight increase of osmolality at 80 h of induction was most likely due to release of cellular compartments of Pichia cells into culture medium. The index of cell lysis at 80 h of induction in this case of the control was 25±5 mg DNA/L (FIGS. 2 B and E). (Note: no data is presented for xylitol at 80 hours of induction because the culture was terminated prior to this time point).

Among sugars and sugar alcohols, maltitol maintained osmolality at higher level (˜600 mOsm/kg H₂O) and reduced cell lysis most significantly (<1 mg DNA/L). The intact GIP in the control was produced 50 mg/L at 58 h of induction but was not observed (˜0 mg/L) at 80 h of induction due to most likely proteolytic degradation caused by cell lysis. The intact GIP in supplementation of matitol was produced 100 mg/L at 80 h of induction, which its volumetric productivity is 2-fold higher than that of control.

The osmolalities in arabinose, ribose and myo-inositol were slightly lower than that of maltitol, but the cell lysis indexes were much lower than that of control (25±5 mg DNA/L). The range of concentration is 25-50 g/L for each sugar or sugar alcohol. By supplementation of osmoprotectants in batch medium, the induction of intact GIP in glycol-engineered P. pastoris was extended to at least 1 d and the volumetric productivity increased in 2-fold than the control without supplementation of osmoprotectent.

### Example 4

### Fermentability of Sorbitol and Maltitol Determined by HPLC

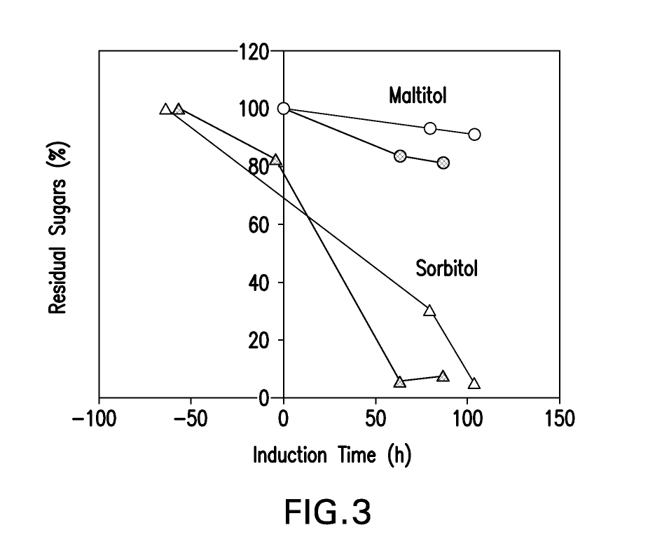

The P. pastoris YGLY 21058 strain was cultivated in 1 L bioreactor and 50 g/L of maltitol was mixed in BSGY medium. The samples were taken at the beginning of fermentation, at the end of glycerol fed-batch before adding maltitol, and two different time-points in methanol induction phase, as shown in FIG. 3. The supernatant was separated by the centrifugation at 13,000 rpm for 1 minutes using micro-centrifuge. The samples properly diluted with distilled water were filtered using 0.2 μm-filter and the residual concentrations of sorbitol and maltitol were determined by HPLC using AMINEX HPX-87H column. The residence times (RTs) for sorbitol and maltitol were 11.5 and 9.3 min respectively. The data were shown in FIG. 3 as two independent runs of cultivation.

The initial sorbitol in batch medium of BSGY began to be consumed after batch growth phase and was actively metabolized by P. pastrois in methanol induction phase. However 50 g/L of maltitol in batch medium was not consumed but its level tended to be slightly declined due to dilution with the volume of methanol fed into fermentor.

This result demonstrated that maltitol is non-fermentable sugar alcohol for P. pastoris but sorbitol is highly fermentable carbon source as shown in FIG. 3.

### Example 5

### Effect of Media Supplements on Cell Robustness of a P. pastoris Production Strain Producing an Antibody Fc Fragment

Using a P. pastoris strain (YGLY27893) which produces an antibody Fc-fragment, the osmoprotective effect of combining sorbitol and maltitol was tested as follows: sorbitol(−) maltitol(−); sorbito(+) maltitol(−); sorbitol(1) maltito(+). The concentrations of sorbitol and maltitol were 18 g/L and 50 g/L respectivetly. (Note: Sorbitol(+) maltito(−) is identical to BSGY medium).

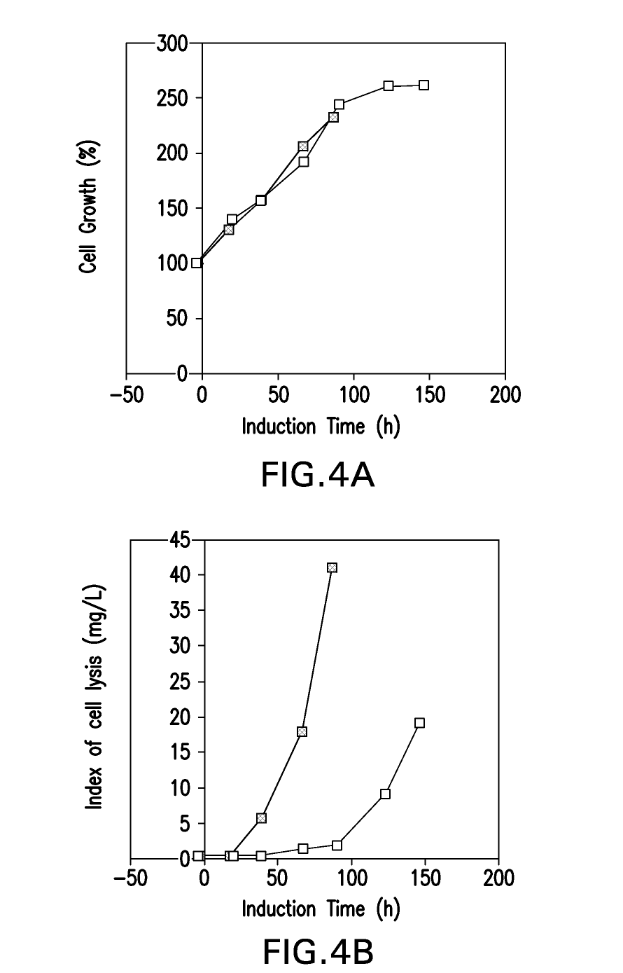

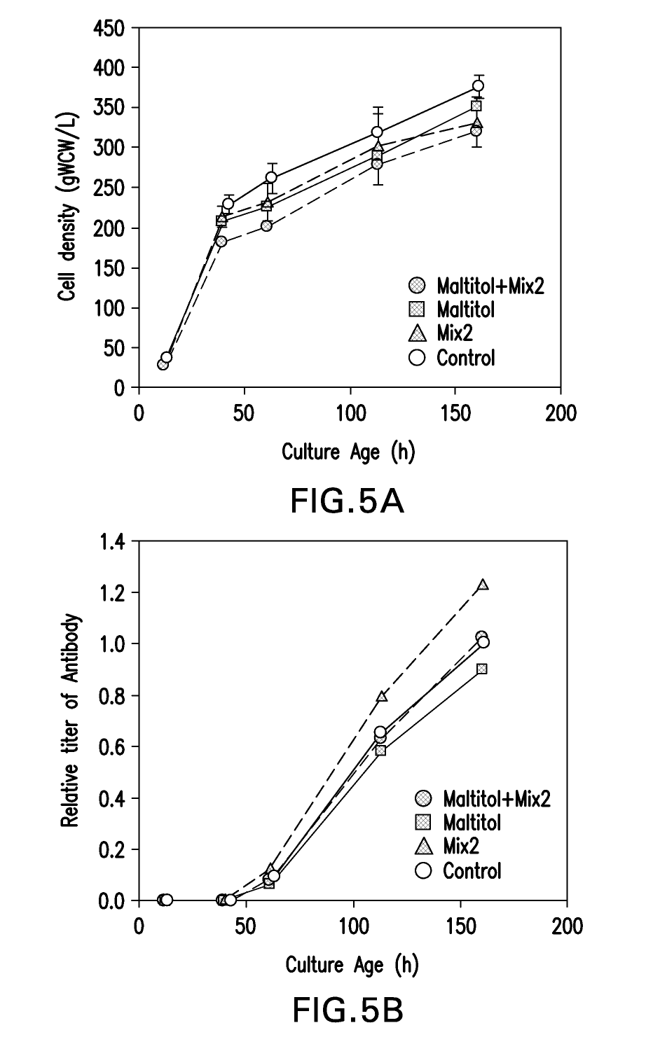

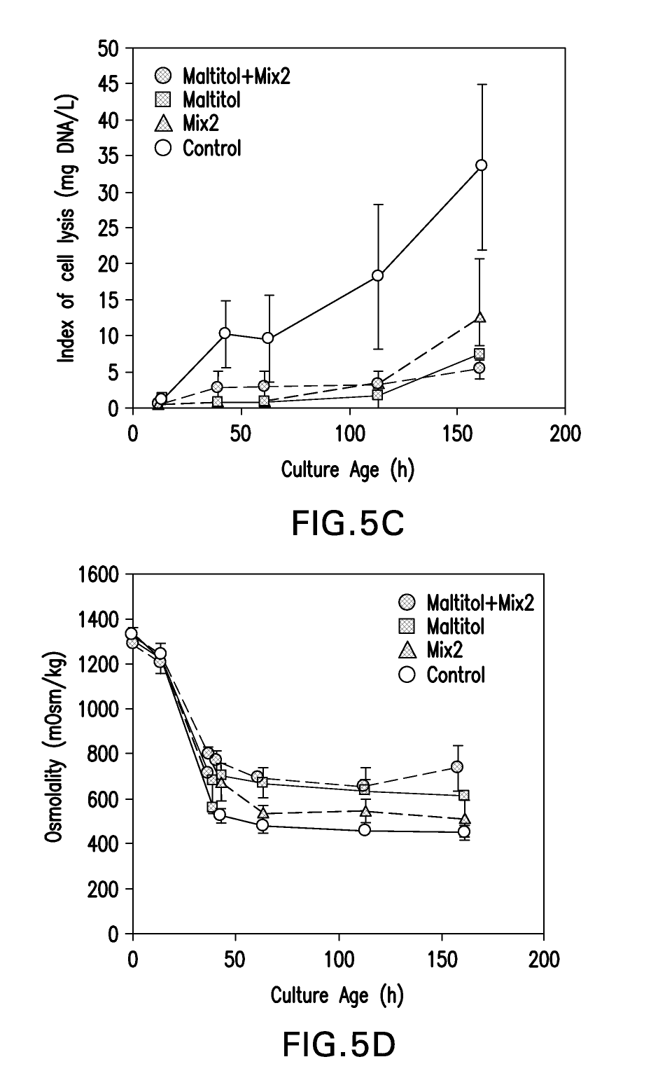

The cell growth was expressed as [Cell (t)]/[Cell (0)]×100(%), here, [Cell (t)] is the wet cell weight at t (h) of induction and [Cell (0)] is the wet cell weight at 0 (h) of induction, because the cell density at the beginning of induction in each case was different due to absence or presence of sorbitol as a fermentable sugar. The cell was heavily lysed around 80 h of induction in BSGY medium without maltitol, whereas the cell was continuously grown for 150 h of induction in BSGY medium with maltitol (FIG. 4). I re-draw the FIG. 4 as shown in the last page (p 47 after FIG. 5).

The osmoprotective effect of maltitol (50 g/L) in combination with a supplement comprising a mixture of nutrients (Mix2) consisting of amino acid (AA1), vitamins (Vit4), trace metal ions (TM4), and basal salts (BSM2) as described in Table 2 was also evaluated using a glycoengineered P. pastoris (YGLY13979) strain producing a monoclonal antibody was cultivated in 1 L bioreactors (Biostat Q-plus, Satorius, Germany).

TABLE 2
Components of Mix2

| Solutions | | Concentration in |
|---|---|---|
| of Mix2 | Components | stock |
| | | g/L |
| AA1 | Alanine | 3.4 |
| | Arginine | 3.4 |
| | Asparagine | 3.8 |
| | Aspartate | 3.4 |
| | Cysteine | 3.8 |
| | Glutamate | 3.4 |
| | Glutamine | 3.4 |
| | Glycine | 3.4 |
| | Histidine | 4.2 |
| | Isoleucine | 3.4 |
| | Leucine | 6.8 |
| | Lysine | 3.4 |
| | Methionine | 4.2 |
| | Phenylalanine | 3.4 |
| | Proline | 3.4 |
| | Serine | 3.4 |
| | Threonine | 3.4 |
| | Tryptophan | 4.2 |
| | Tyrosine | 3.4 |
| | Valine | 3.4 |
| | | mM |
| Vit4 | D-biotin | 5 |
| | D-pantothenic acid hemicalcium salt | 5 |
| | Nicotinic acid | 200 |
| | Para-aminobenzoic acid | 100 |
| | Pyridoxine | 200 |
| | Riboflavin | 5 |
| | Thiamin hydrochloride | 5 |
| | Ascorbic acid | 50 |
| | | g/L |
| TM4 | Boric acid (H₃BO₃) | 0.2 |
| | Ferrous sulfate (FeSO₄•H₂O) | 14.0 |
| | Manganese sulfate (MnSO₄•H₂O) | 0.5 |
| | Potassium iodide (KI) | 0.2 |
| | Sodium molybdate (Na₂MoO₄•2H₂O) | 0.24 |
| | Zinc chloride (ZnCl₂) | 5.0 |
| BSM2 | Phosphoric acid (H₃PO₄) | 26.7 |
| | Calcium sulfate (CaSO₄) | 0.93 |
| | Potassium sulfate (K₂SO₄) | 18.2 |
| | Magnesium chloride (MgCl₂) | 12.3 |
| | Potassium hydroxide (KOH) | 4.13 |

A P. pastoris YGLY13979 producing a monoclonal antibody was cultivated in 1 L bioreactors (Biostat Q-plus, Satorius, Germany). For fermentation in 1 L bioreactor, a vial (1 mL) of RCB (Research Cell Bank) was inoculated into 200 mL of BSGY medium in 1 L-baffled flask. The culture incubated at 24° C., while shaking on an orbital shaker at 180 rpm for 48±4 h. The bioreactor was inoculated with a 10% volumetric ratio of seed to initial BSGY medium. Cultivation conditions were following: temperature set at 24±0.5° C., pH controlled at 6.5±0.1 with 30% ammonium hydroxide, dissolved oxygen was maintained at 20% of saturation by cascading agitation rate on the addition of pure oxygen to the fixed airflow rate of 0.7 vvm. The fermentation profile of the production strain was examined using four different cultivation conditions after batch phase.

For the control, a 50% glycerol solution containing 12.5 mL/L of PTM1 salts (6.5 g FeSO₄.7H₂O, 2.0 g ZnCl₂, 0.6 g CuSO₄.5H₂O, 3.0 g MnSO₄.7H₂O, 0.5 g CoCl₂.6H₂O, 0.2 g NaMoO₄.2H₂O, 0.2 g biotin, 80 mg NaI, 20 mg H₃BO₄ per L) was fed constantly at an initial rate of 0.08 h⁻¹ for 8 h After depletion of the initial glycerol (4%).

For Mix2, 10 mL AA1, 4 mL Vit4, 4 mL TM4, and 4 mL BSM2 were mixed with 62.5 mL of 80% glycerol and 15.5 mL of water. The Mix2 solution was fed during the glycerol fed-batch period instead of regular glycerol solution, which was used for the control.

For Maltitol, 43.5 mL of 80% matitol solution was added into bioreactor to achieve 50 g/L concentration at the end of glycerol fed-batch phase.

For Maltitol+Mix2, two conditions of Maltitol and Mix2 were combined.

Induction was initiated after a 30 min starvation phase when methanol was fed. Methanol containing 12.5 mL/L of PTM1 salts (6.5 g FeSO₄.7H₂O, 2.0 g ZnCl₂, 0.6 g CuSO₄.5H₂O, 3.0 g MnSO₄.7H₂O, 0.5 g CoCl₂.6H₂O, 0.2 g NaMoO₄.2H₂O, 0.2 g biotin, 80 mg NaI, 20 mg H₃BO₄ per L) was fed constantly starting at 1.33 g/L/h under methanol limited condition. Agitation speed was changed from cascade mode with agitation speed and pure oxygen.

The Mix2 significantly reduced the cell lysis (FIG. 5 C) and improved the titer of antibody (1.2-fold). Maltitol only and Maltitol+Mix2 reduced the cell lysis significantly with slight reduction of cell yield.

The osmolality of control in BSGY medium without any supplementations was decreased down to 470±30 mOsm/kg during methanol induction phase but cell lysis index significantly increased from 10 mg DNA/L at 4 h of induction to 35 mg DNA/L at 125 h of induction. The osmolality in methanol phase increased up to 50 and 100 mOsm/kg when supplemented with Mix2 and Maltitol, respectively. The cell lysis was significantly reduced when Mix2 and maltitol were supplemented by either individual addition or combination (FIG. 5).

At least 50 mOsm/kg higher osmolality than the control (BSGY without supplementation) seemed to prevent the cell from the cell lysis. The minimum concentration of nutrients supplemented in BSGY medium is required to increase and maintain the osmolality of medium during the induction phase. For instance, the effective range of concentration of maltitol in BSGY medium was higher than 25 g/L. More desirable range is 25-50 g/L. The inclusion of the osmoprotectant has no effect on the presence of host cell proteins and on the product quality such as N-glycosylation of the target proteins.

Patents, patent applications, publications, product descriptions, and protocols are cited throughout this application, the disclosures of which are incorporated herein by reference in their entireties for all purposes. All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. §1.57(b)(1), to relate to each and every individual publication, database entry (e.g. Genbank sequences or GeneID entries), patent application, or patent, each of which is clearly identified in compliance with 37 C.F.R. §1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

### REFERENCES

Arakawa T, Tsumoto K, Kita Y, Chang B, anf Ejima D. Biotechnology applications of amino acids in protein purification and formulation. Amino Acids (2007); 33:587-605.
Blackwell J R, Horgan R. A novel strategy for production of a highly expressed recombinant protein in an active form. FEBS Letters (1991) 295 (1,2,3): 10-12
Boniolo F S, Rodrigues R C, Delatorre E O, Da Silveira M M, Flores V M Q, Berbert-Molina M A. Glycine betaine enhances growth of nitrogen-fixing bacteria Gluconacetobacter diazotrophicus PALS under saline stress conditions. Curr. Microbiol. 2009 December; 59(6):593-9
Catena R, Larzabal L, Larrayoz M, Molina E, Hermida J, Agorreta J, Montes R, Pio R, Montuenga L M, Calvo A. VEGF₁₂₁b and VEGF₁₆₅b are weakly angiogenic isoforms of VEGF-A. Mol Cancer. 2010 Dec. 31; 9:320
Cereghino, J T, Cregg, J M. Heterologous protein expression in the methyltrophic yeast Pichia pastoris. FEMS Microbiology Reviews (2000); 24: 45-66
Charoenrat, T et al., Oxygen-limited fed-batch process: An alternative control for Pichia pastoris recombinant protein proceses. Bioprocess Biosyst. Eng. (2005) 27(6):399-406
Choi, D B and Park E Y. Process Biochemistry (2006) 41:390-397
Daly R, Hearn M T W. Expression of heterologous proteins in Pichia pastoris: a useful experimental tool in protein engineering and production. J Mol. Recognit. 2005; 18:119-38.
d'Anjou, M C and Daugulis, A J. A rational approach to improving productivity in recombinant Pichia pastoris fermentation. Biotech. And Bioeng. (2001) 72(1):1-11
Dominguez-Ferreras A, Pérez-Arnedo R, Olivares J, Sanjuán J. Importance of trehalose biosynthesis for Sinorhizobium meliloti Osmotolerance and nodulation of Alfalfa roots. J Bacteriol. 2009 December; 191(24):7490-99
Damasceno L M et al., An optimized fermentation process for high level production of a single-chain Fv antibody fragment in Pichia pastoris. Potein Expr. Purif. 37(1):18-26 (2004)
Ellis S B, Brust P F, Koutz P J, Waters A F, Harpold M M, Gingeras T R. Isolation of alcohol oxidase and two other methanol regulatable genes from the yeast Pichia pastoris. Mol. Cell. Biol. 1985 May; 5(5):1111-21.
Gigout A, Buschmann M D, Jolicoeur M. The fate of Pluronic F-68 in Chondrocytes and CHO cells. Biotechnol. Bioeng. Aug. 1, 2008: 100(5):975-87.
Hamilton S R, Gerngross T U. Glycosylation engineering in yeast: the advent of fully humanized yeast. Curr Opin Biotechnol. 2007; 18(5):387-92. PMID: 17951046.
Han Y K, Koo T Y, Lee G M. Enhanced interferon-β production by CHO cells through elevated osmolality and reduced culture temperature. Biotechnol. Prog. 2009; 25(5):1440-47.
Hohmann, S. Osmotic stress signaling and osmoadaptation in yeasts (2002) Microbiol. Mol. Biol. Reviews 66 (2):300-372
Kiewietdejonge A, Pitts M, Cabuhat L, Sherman C, Kladwang W, Miramontes G, Floresvillar J, Chan J, Ramirez R M. Hypersaline stress induces the turnover of phosphatidylcholine and results in the synthesis of the renal osmoprotectant glycerophosphocholine in Saccharomyces cerevisiae. FEMS Yeast Res. 2006 March; 6(2):205-17
Kim T K, Ryu J S, Chung J Y, Kim M S, Lee G M. Osmoprotective effect of glycine betaine on thrombopoietin production in hyperosmotic Chinese Hamster Ovary cell culture: clonal variations. Biotechol. Prog. 2000; 16:775-81.
Oyaas, K et al. Hyperosmotic hybridoma cell cultures: increased monoclonal antibody production with addition of glycine betaine. Biotechnol. Bioeng (1994) 44:991-998.
Pacis E, Yu M, Autsen J, Bayer R, Li F. Effect of cell culture conditions on antibody N-linked glycosylation—what affects high mannose 5 glycoform. Biotechnol. Bioeng. 2011 October; 108(10):2348-58.
Potgieter T I, Cukan M, Drummond J E, Houston-Cummings N R, Jiang Y, Li F, Lynaugh H, Mallem M, McKelvey T W, Mitchell T, Nylen A, Rittenhour A, Stadheim T A, Zha D, d'Anjou M. Production of monoclonal antibodies by glycoengineered Pichia pastoris. J Biotechnol. 2009; 139(4):318-25.
Ramon, R et al., Sorbitol co-feding reduces metabolic burden caused by the overexpression of a Rhizopus oryzae lipae in Pichia pastoris. J. Biotechnol. (2007) 130:39-46
Ryu J S, Kim T K, Chung J Y, Lee G M. Osmoprotective effect of glycine betaine on foreign protein production in hypeosmotic recombinant Chinese Hamster Ovary cell Cultures differs among cell lines. Biotechnol. Bioeng. 2000 Oct. 20; 70(2):167-75.
Shi, X et al., Optimal conditions for the expression of single-chain antibody (scFv) gene in Pichia pastoris. Protein Expression & Purification (2003) 28:321-330
Stergiou C, Zisimopoulou P, Tzartos S J. Expression of water-soluble, ligand-binding concatameric extracellular domains of the human neuronal nicotinic receptor alpha4 and beta2 subunits in the yeast Pichia pastoris: glycosylation is not required for ligand binding. J Biol Chem. 2011 March 18; 286(11):8884-92
Van der Heide T, Poolman B. Osmoregulated ABC-transport system of Lactococcus lactis senses water stress via changes in the physical state of the membrane. Proc Natl Acad Sci USA. 2000 Jun. 20; 97(13):7102-6.
Vozza L A, Wittwer L, Higgins D R, Purcell T J, Bergseid M, Collins-Racie L A, LaVallie E R, Hoeffler J P. Production of a recombinant bovine enterokinase catalytic subunit in the methylotrophic yeast Pichia pastoris. Biotechnology (N Y). 1996 January; 14(1):77-81.
Ye, Jianxin et al. Optimization of a glycogengineered Pichia pastoris cultivation process for commercial antibody production. Biotechnol. Prog. 2011, 27(6):1744-1750.
Zanghi J A, Schmelzer A E, Mendoza T P, Knop R H, Miller W M. Bicarbonate concentration and osmolality are key determinants in the inhibition of CHO cell polysialylation under elevated pCO2 or pH. Biotechnol. Bioeng. 1999 Oct. 20; 65(2):182-91.
Zhang, W, et al. Fermentation strategies for recombinant protein expression in the methylotrophic yeast Pichia pastoris. Biotechnol. Bioprocess Eng. (2000) 5:275-287

1. A cell culture medium for the production of a protein of interest in yeast host cells using a fed-batch fermentation process wherein the cell culture medium comprises a basal medium supplemented with an osmoprotectant.

2. The culture medium of claim 1, wherein the osmoprotectant is selected from a nonfermentable sugar and a nonfermentable sugar alcohol.

3. The culture medium of claim 2, wherein the osmoprotectant is selected from maltose, sorbose, ribose, maltitol, myo-inositol, mellibiose, and quinic acids.

4. The culture medium of claim 3, wherein the osmoprotectant is present at a concentration of about 25 g/L to about 50 g/L.

5. The culture medium of claim 3, wherein the osmoprotectant increases the osmolarity of the batch media to more than about 50 mOsm/kg compared to the osmolarity of a fed-batch culture of the same host cell in culture media not supplemented with an osmoprotectant.

6. The culture medium of claim 2, wherein the basal medium is selected from BSGY which is optionally supplemented with amino acids, basal salts, vitamins, and trace metals.

7. The culture medium of claim 2, wherein the basal medium is BSGY and the osmoprotectant is maltitol.

8. A methanol fed-batch fermentation medium comprising an osmoprotectant selected from a nonfermentable sugar and a nonfermentable sugar alcohol.

9. The methanol fed-batch fermentation medium of claim 8, wherein the osmoprotectant is selected from maltose, sorbose, ribose, maltitol, myo-inositol, mellibiose, and quinic acids.

10. The fed-batch medium of claim 9, wherein the osmoprotectant is present at a concentration of about 25 g/L to about 50 g/L.

11. A method of improving the volumetric productivity of a glycoprotein of interest in a yeast fermentation culture comprising:
a) providing a glycerol fed-batch yeast host cell culture comprising high density yeast cells that contain a gene encoding a polypeptide of interest, which gene is expressed under conditions of fermentation;
b) providing a methanol fed-batch medium containing an osmoprotectant; and
b) inducing the yeast host cells under fermentation conditions that allow expression of the recombinant protein wherein the volumetric productivity of the protein of interest is higher than the productivity obtained using identical fermentation conditions to produce the same glycoprotein in medium that lacks the osmoprotectant.

12. The method of claim 11, wherein the yeast cells are Pichia host cells selected from cells are glycoengineered to:
a) include a nucleic acid that encodes an alpha-1,2-mannosidase that has a signal peptide that directs it for secretion;
b) comprise a nucleic acid sequence that encode one or more glycosylation enzymes or oligosaccharyltransferases;
b) comprise a disruption or deletion of one or more of a functional gene product encoding an alpha-1,6-mannosyltransferase activity, mannosylphosphate transferase activity, a β-mannosyltransferase activity or a dolichol-P-Man dependent alpha(1-3) mannosyltransferase activity; and
c) produce glycoproteins that have predominantly an N-glycan selected from the group consisting of complex N-glycans, hybrid N-glycans, and high mannose N-glycans.

13. The method of claim 12, wherein the gene encoding a polypeptide of interest encodes a therapeutic protein.

14. The method of claim 13, wherein the therapeutic protein is selected from the group consisting of kringle domains of the human plasminogen, erythropoietin, cytokines, coagulation factors, soluble IgE receptor α-chain, IgG, IgG fragments, IgM, urokinase, chymase, urea trypsin inhibitor, IGF-binding protein, epidermal growth factor, growth hormone-releasing factor, annexin V fusion protein, angiostatin, vascular endothelial growth factor-2, myeloid progenitor inhibitory factor-1, osteoprotegerin, α-1 antitrypsin, DNase II, α-feto proteins, insulin, Fc-fusions, and HSA-fusions.

15. A method for producing glycoprotein compositions in Pichia sp host cells comprising growing host cells of claim 13 under inducing conditions.

16. A method of improving the cell viability of engineered Pichia strains comprising:
a) providing a high density Pichia cell culture wherein the cells contain a gene encoding a polypeptide of interest, which gene is expressed under conditions of fermentation;
b) providing a methanol fed-batch medium containing an osmoprotectant; and
b) inducing the Pichia cells under fermentation conditions that allow expression of the recombinant protein wherein the cell viability of the Pichia cells is greater than the viability of identical Pichia cells cultured under identical fermentation conditions in medium lacking the osmoprotectant.

17. The method of claim 15, wherein the osmoprotectant is selected from a nonfermentable sugar and a nonfermentable sugar alcohol.

18. The method of claim 17, wherein the osmoprotectant is selected from maltose, sorbose, ribose, maltitol, myo-inositol, mellibiose, and quinic acids.

19. The method of claim 18, wherein the osmoprotectant is present at a concentration of about 25 g/L to about 50 g/L.
